(19) United States
(12) Patent Application Publication
(10) Pub. No.: US 2014/0121607 A1
(43) Pub. Date: May 1, 2014

(54) SWITCHABLE DEVICE FOR CLOSED SUCTION CATHETERS AND SYSTEMS THEREOF

(71) Applicant: LILY MEDICAL CORPORATION, Miao-Li Hsien (TW)

(72) Inventor: WEI HSUAN CHANG, MIAOLI COUNTY (TW)

(73) Assignee: LILY MEDICAL CORPORATION, MIAO-LI HSIEN (TW)

(21) Appl. No.: 13/867,074

(22) Filed: Apr. 20, 2013

(30) Foreign Application Priority Data
Oct. 29, 2012 (TW) 101220873

(51) Int. Cl. A61M 39/22; A61M 1/00

(52) U.S. Cl. CPC A61M 39/22; A61M 1/00
USPC 604/246

(57) ABSTRACT

A switchable device for closed suction catheters includes a device body and a two-way switch valve. The device body is formed with a valve sleeve, a patient tubing adaptor, a side port, and a catheter adaptor. The valve sleeve is formed with a cavity therein to allow the two-way switch valve to correspondingly rotate with respect to the device body. The catheter adaptor and the patient tubing adaptor are formed on opposing ends of the valve sleeve, thereby able to establish an unobstructed path therebetween. The side port is slantingly formed and extended from the patient tubing adaptor for smooth passage of flow therebetween. A groove is formed on one end of the patient tubing adaptor as a 360-degree freely rotatable coupling element thereof.

### BACKGROUND OF THE INVENTION

1. Field of the Invention

The instant disclosure relates to a switchable device for close suction catheters and systems thereof; in particular, to a device having a 360-degree rotatable tubing, and a two-way switch valve that allows a user to quickly and effectively switch directions of flow for secretion suctioning and providing oxygen supply to patients.

2. Description of Related Art

Close suction catheters differs from regular catheters in which the prior can perform the suctioning or aspiration procedure on a patient while maintaining the oxygenation and ventilation to patients without removing the tubing to supply oxygen or ventilation, thereby effectively reducing the chances of affecting the critical oxygen supply to patients, preventing the patients from respiratory infections and the environment from exposure to infectious materials.

Referring to FIG. 1, during the suctioning procedure, a conventional closed suction catheter 1a is typically connected to the patient through a fitting 11a. However, the conventional catheter 1a lacks a device providing an opening and closing control which prevents the materials which flow through a catheter 12a, a tubing 13a, and a ventilation tubing 14a to intermix, thereby rendering inconvenience and additional risks on the patients. To prevent such inconvenience, an additional flow control device (not shown in figure) can be disposed between the fitting 11a, the catheter 12a, and the tubing 13a to control flow therebetween. However, such device requires at least three isolated valves and has a complicated structure. Thus, the conventional device increases production cost and functional inconvenience.

Furthermore, in common suctioning procedures, physicians usually consider the safer application of catheters to help patients remove secretions from the airways especially for patients who are not capable of removing the secretions themselves. During the suctioning procedure, the catheter must be advanced into the trachea, reached the intersection of the trachea and the bronchus of the patient, and then withdrawn approximately one to two centimeters in order to perform suctioning. As a resulting, such procedure can cause discomfort in patients, and increase the chance of infections caused by bacteria. In addition, since the procedure requires moving of the trachea, such actions may lead to cardiac arrhythmia, slowing of the heart, or heart failure which may directly endanger the lives of the patients.

To address the above issues, the inventor strives via associated experience and research to present the instant disclosure, which can effectively improve the limitation described above.

### SUMMARY OF THE INVENTION

The instant disclosure provides a switchable device for closed suction catheter systems for facilitating medical staffs to quickly and effectively change fluid flow via a two-way switch valve. Through a 360-degree of free rotational tubing, the instant disclosure is compatible with the various types of medical environment and effectively enhances the setup speed to alleviate the burden on the patients. The switchable device includes a device body integrally formed with a valve sleeve defining a cavity interconnected with an opening arranged proximate to the top of the valve sleeve. A patient tubing adaptor is formed on and interconnected with a side of the valve sleeve and is formed with a groove on an end of the patient tubing adaptor to engage and freely rotate with respect to a patient end adaptor. A side port is slantingly extended from and interconnected with the patient tubing adaptor, and a catheter adaptor is formed on and interconnected with a side opposing the patient tubing adaptor of the valve sleeve. A two-way switch valve is defined by a stem and a knob and is engaged to the device body in a freely rotatable fit. The stem is securely disposed in the cavity and formed with a first opening and a second opening to communicate flow therebetween. The knob is integrally formed with a plurality of handles and is generally arranged on the apex of the stem.

Moreover, the switchable device may also include a catheter sleeve and a press control valve to form a closed suction catheter system.

What is noteworthy are the junction formed between the patient tubing adaptor and the catheter adaptor of the switchable device via the two-way switch valve, and the side port formed slantingly on the patient tubing adaptor which lower production cost and enhance efficiency of fluid flow control. In addition, the switchable device is compatible with various medical environments due to the 360-degree freedom of rotation from the structural design of the patient tubing adaptor and the patient end adaptor. Furthermore, the closed suction catheter system includes an indicator which is selectively paired with a plurality of depth markings formed on the catheter. Successively, the catheter can advance into an airway of the patient with a pre-determined distance in a safe and reduced infectious fashion.

In order to further understand the instant disclosure, the following embodiments and illustrations are provided. However, the detailed description and drawings are merely illustrative of the disclosure, rather than limiting the scope being defined by the appended claims and equivalents thereof.

### BRIEF DESCRIPTION OF THE DRAWINGS

FIG. 1 is a perspective view of a conventional closed suction catheter device;

FIG. 2 is a perspective view of the switchable device according to an embodiment of the instant disclosure;

FIG. 3 is an exploded view of the switchable device according to the embodiment of the instant disclosure;

FIG. 4A is a schematic diagram of the switchable device according to the embodiment of the instant disclosure;

FIG. 4B is a second schematic diagram of the switchable device according to the embodiment of the instant disclosure;

FIG. 5A is a third schematic diagram of the switchable device according to the embodiment of the instant disclosure;

FIG. 5B is a fourth schematic diagram of the switchable device according to the embodiment of the instant disclosure; and

FIG. 6 is a perspective view of the closed suction catheter system according to the embodiment of the instant disclosure.

### DETAILED DESCRIPTION OF THE PREFERRED EMBODIMENTS

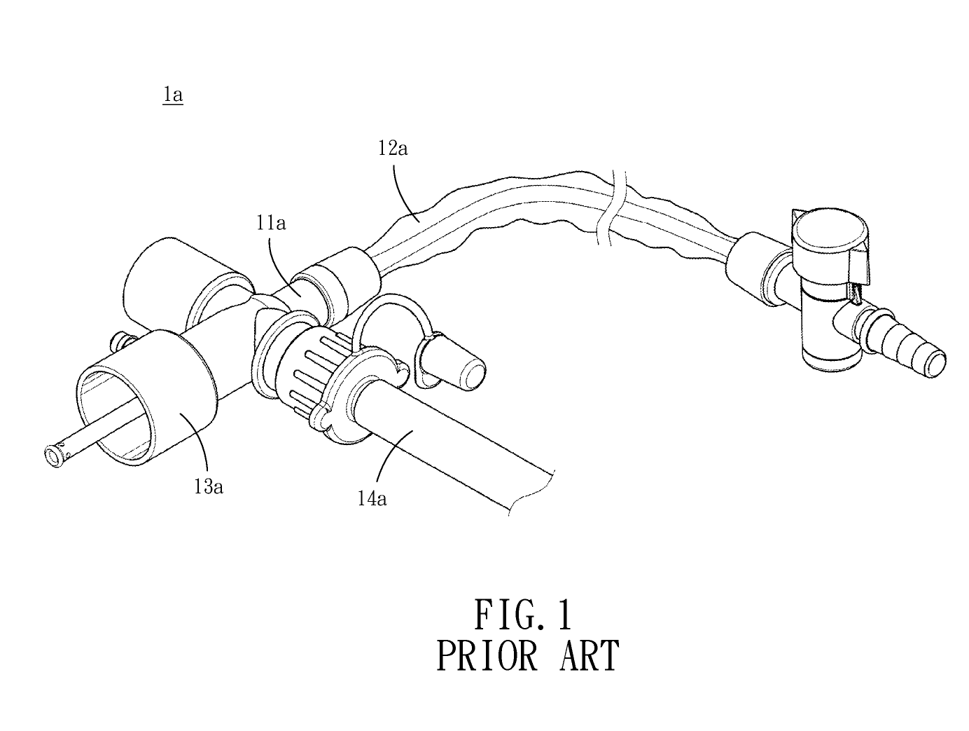

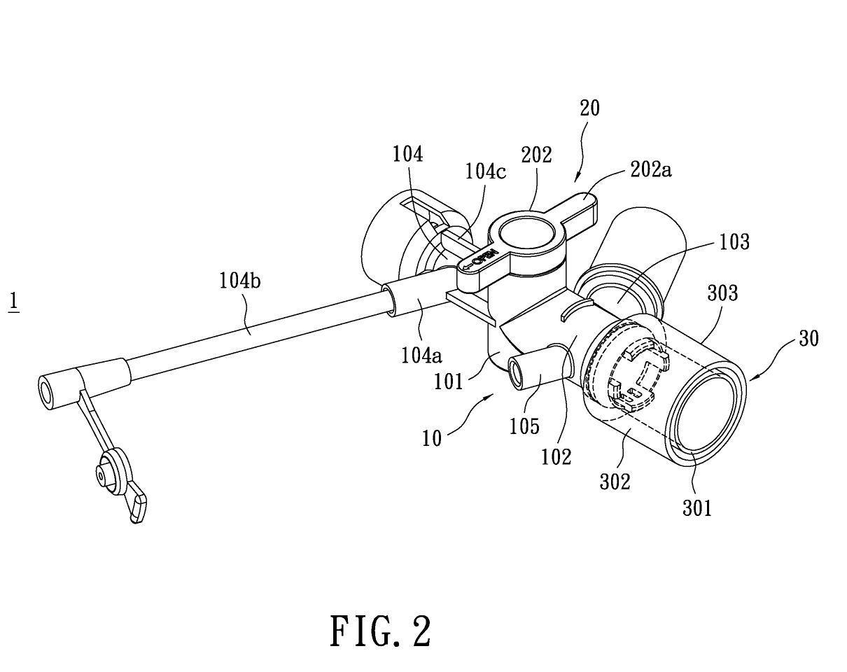

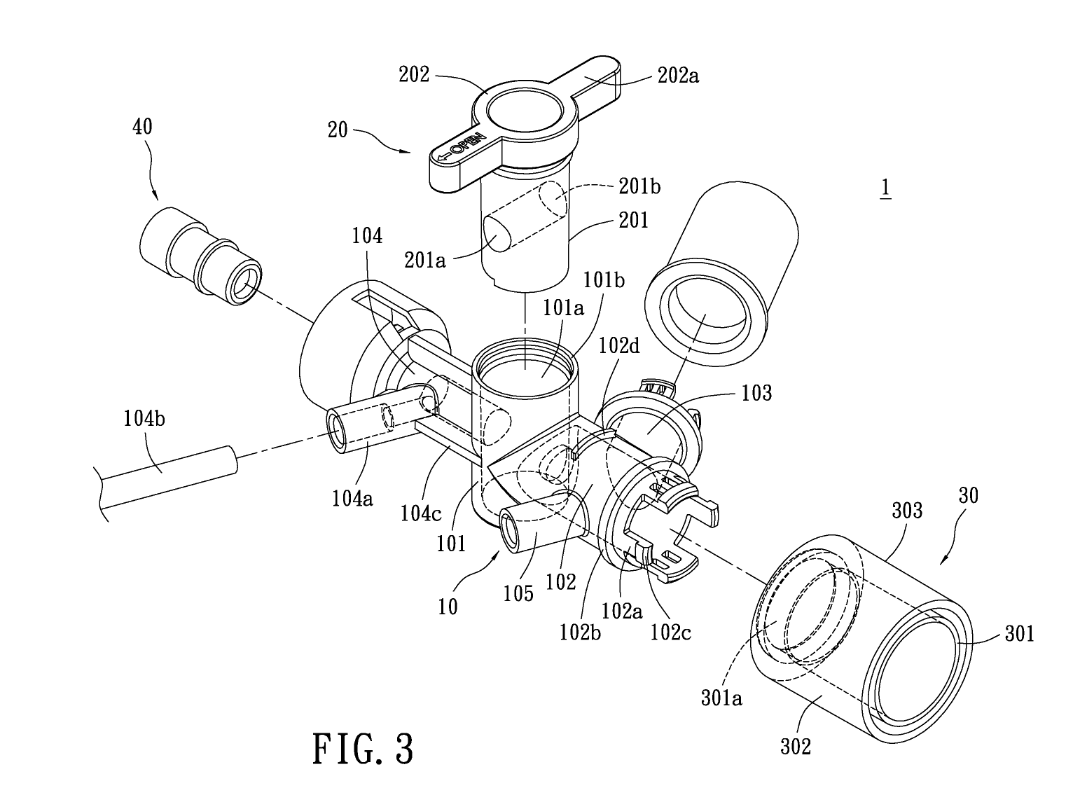

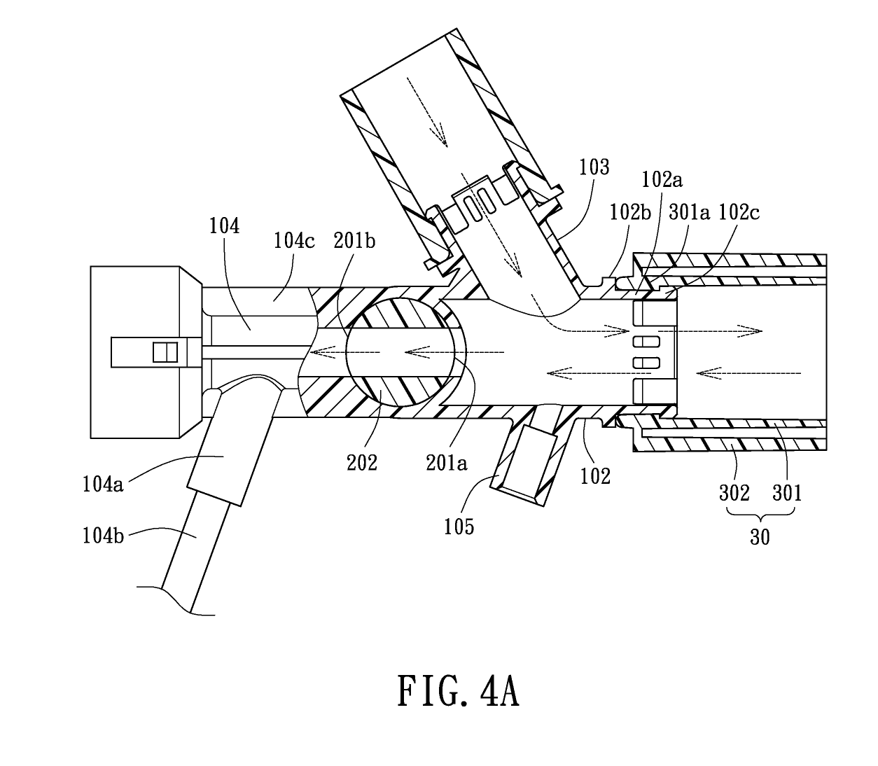

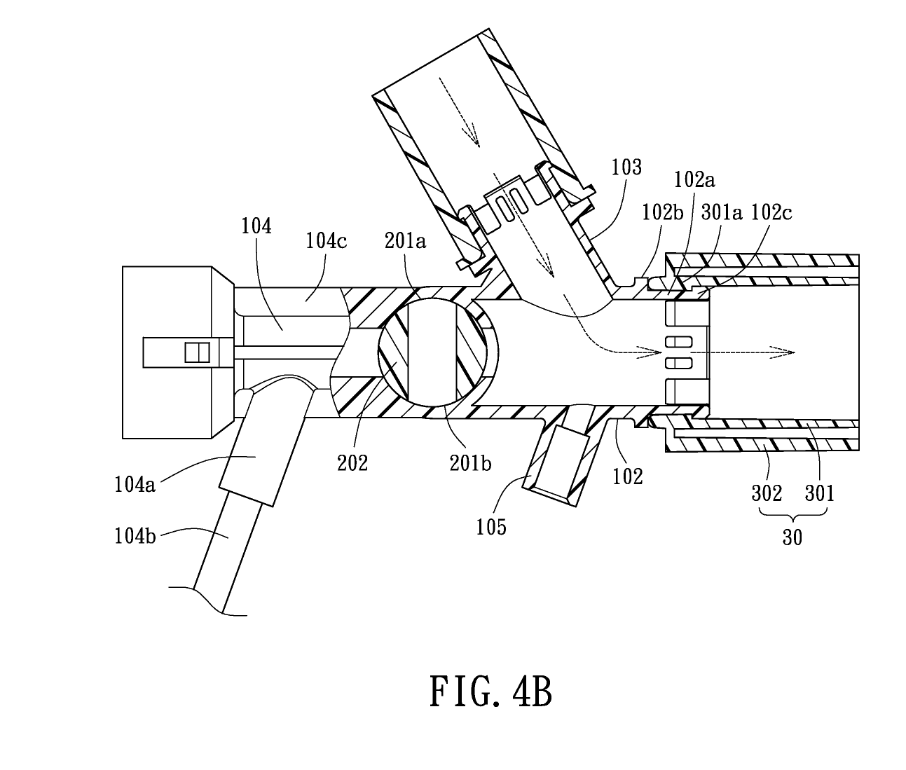

The instant disclosure relates to a switchable device 1 for closed suction catheter systems to aspirate secretions from a patient defined as a distal end while maintaining respiratory support to the patient via a closed suction catheter system defined as a proximal end, ventilation tubing, and related components. Referring to FIGS. 2 and 3, the device 1 includes a device body 10, and a two-way switch valve 20. The device 1 includes and is formed integrally with a valve sleeve 101, a patient tubing adaptor 102, a side port 103, and a catheter adaptor 104 to effectively prevent incidence of secondary and viral infections. As illustrated in FIG. 3, the patient tubing adaptor 102 and the catheter adaptor 104 are formed on opposite sides of the valve sleeve 101. The valve sleeve 101 defines a cavity 101a for communicating fluids between the patient tubing adaptor 102 and the catheter adaptor 104, and for conformingly retaining a two-way switch valve 20 in the cavity 101a. The valve sleeve 101 also defines an opening 101b. The side port 103 is slantingly formed at an obtuse angle and extended from the patient tubing adaptor 102 for smoothly communicating gas flow therebetween without restrain, thus facilitates breathing for tracheostomy patients and reduces the chance for accidents. Moreover, the two-way switch valve 20 accurately controls fluid flow directions such as communicating fluid flow between the patient tubing adaptor 102 and the catheter adaptor 104 or communicating fluid flow between patient tubing adaptor 102 and the side port 103 for preventing fluid flow between the side port 103 and the cavity 101a via a misalignment design (as illustrated in FIG. 4A and 4B). As a result, the conventional three-way valve (referring to FIG. 1) can be eliminated, production cost can be effectively reduced and the efficiency of fluid control can be improved.

The two-way switch valve 20 includes a knob 202 and a stem 201 while rotationally engaged to the device body 10. The stem 201 is conformingly disposed in the cavity 101a of the valve sleeve 101 through the opening 101b. Moreover, the stem 201 defines a first opening 201a and a second opening 201b forming on opposing sides of the stem 201 thereby forming a channel for communicating fluids therein. The proximal end of the patient tubing adaptor 102 is correspondingly arranged to communicate flow with the first opening 201a, the distal end of the catheter adaptor 104 is correspondingly arranged to communicate flow with the second openings 201b or the vice versa, thereby establishing an inline configuration for communicating flow between the first and second openings 201a, 201b, the patient tubing adaptor 102 and the catheter adaptor 104. The knob 202 is formed with a plurality of handles 202a extending therefrom for facilitating turning of the knob 202 and is integrally formed on the apex of the stem 201. Furthermore in FIG. 3, the patient tubing adaptor 102 of the device body 10 is coupled with a patient end adaptor 30. The patient tubing adaptor 102 defines a groove 102a forming around the periphery of the tubing adaptor 102 to allow a 360-degree freedom of rotation with the patient end adaptor 30 while coupled therewith. While suitable in all kinds of medical environment, the freedom of rotation prevents accidental suction loss caused by obstruction of flow when patients flip and turn. To summarize the aforementioned, the device 1 is functionally convenient wherein the knob 202 enables the two-way switch valve 20 to be rotated respectively with the device body 10, rendering the catheter adaptor 104 to have an open state, thus allowing the side port 103 to supply oxygen or ventilation to the patient while secretions are being aspirated. By closing the two-way switch valve 20 after aspiration, the catheter adaptor 104 is isolated from the patient end while maintaining the PEEP (positive end expiratory pressure) level of the patient without being affected by a suctioning force from a vacuum source (not shown in figures) at the proximal end. The two-way switch valve 20 also prevents backwash of secretions to the patient end. The instant disclosure is conveniently produced by forming the generally hollowed device body 10 having two open ends and the two-way switch valve 20 conformingly and rotatably arranged within the body 10 to facilitate control of the fluid flow within the catheter adaptor 104, thus resulting in the structurally simple, easily operable, and low cost switchable device 1.

Turning to FIG. 2 in conjunction with FIG. 3 for the perspective view and the exploded view of the instant embodiment, respectively. Both figures further illustrates the instant embodiment wherein the patient tubing adaptor 102 defines a ring member 102b and a plurality of shoulders 102c forming around the periphery of the patient tubing adaptor 102 near the proximal end. The shoulders 102c are preferably formed by elastic materials. The ring member 102b and the shoulders 102c form the groove 102a which engages within the patient end adaptor 30. Specifically, the patient end adaptor 30 includes two generally hollowed layers, an inner layer 301 and an outer layer 302. The inner layer 301 is formed with a lip 301a therein which conformingly engages with the groove 102a thereby allowing the patient tubing adaptor 102 to conformingly engage while rotate with respect to the patient end adaptor 30. Therefore, the instant disclosure is not limited to a single directional application and is compatible with various types of medical equipment (such as suction machines) in hospitals. To facilitate rotation between the patient tubing adaptor 102 and the patient end adaptor 30, silicone oil can be applied between the lip 301a and the groove 102a for the purpose of electrical insulation, waterproof, and seal strengthening. Additionally, the side port 103 has similar structure as the patient tubing adaptor 102 to enable directionally unrestricted compatibility with medical equipment (such as breathing machine) and prevent restricted ventilation or removal of secretions. As mentioned, arranged opposing from the patient tubing adaptor 102 is the catheter adaptor 104. The catheter adaptor 104 is formed with a flush port 104a, and a flush tubing 104b disposed within the flush port 104a for communicating fluid flow between the catheter adaptor 104, the flush port 104a, and the flush tubing 104b.

Furthermore, the patient end adaptor 30 further includes a transparent housing 303. The patient tubing adaptor 102 is formed with an indicator 102d having a colored signage. The indicator 102d can also be arranged on a tangent, a slot, a protrusion, or as an indicator ring integrally formed with the tubing adaptor 102 or be directly printed in the form of a printing mark on the tubing adaptor 102. While the entire side port 103 and tubing adaptor 102 are preferably produced by transparent materials, portions or the entire transparent housing 303 are preferably produced with transparent materials, and the indicator 102d is preferably a colored protrusion. The first opening 201a and the second opening 201b are arranged on opposing sides of the stem 201 forming a channel to communicate fluids between the first opening 201a, the second opening 201b, the tubing adaptor 102 and the catheter adaptor 104. Two handles 202a are correspondingly formed with the first and second openings 201a, 201b while a directional marking is integrally formed on the apex of the knob 202 for facilitating directional control of fluid flow.

Moreover, the catheter adaptor 104 may be formed with at least one rib 104c arranging on the exterior surface of the catheter adaptor 104 to structurally support the radially retracted design of the catheter adaptor 104, thereby significantly reducing production cost. In the instant embodiment, two radially opposing pairs of ribs 104c are arranged on the exterior surface of the catheter adaptor 104 extending along the entire length thereof. However, the shape, quantity, and arrangement of the ribs 104c can be modified based on the desired design and is not limited to the examples of the instant embodiment provided therein.

Furthermore, the catheter adaptor 104 of the instant embodiment includes a catheter (not shown in figures) having a protective sleeve (not shown in figures) inserted from the proximal end. By rotating the knob 202 to a configuration as illustrated in FIG. 4A, the first and second openings 201a, 201b are correspondingly arranged with the patient tubing adaptor 102 and the catheter adaptor 104 in the inline configuration for communicating flow. Successively, the catheter can be advanced into an airway of the patient through the catheter adaptor 104, the second, first openings 201b 201a, and the patient tubing adaptor 102. Consequently, through the suction provided by the vacuum source (not shown in figures) connecting to the catheter (not shown in figures) at the proximal end, sputum and secretions can be removed from the patient along with a tracheostomy tube (not shown in figures) through the inline configuration. In addition, during the aspiration procedure, the side port 103 continuously supplies oxygen to the patient through the patient tubing adaptor 102. After the aspiration procedure, the catheter is retracted through the patient tubing adaptor 102, the first and second openings 201a, 201b, and then drawn back into the catheter adaptor 104. Thereafter, the knob 202 is rotated through which the patient tubing adaptor 102 is isolated from the catheter adaptor 104 at the patient end as illustrated in FIG. 4B. As a result, PEEP (positive end expiratory pressure) level of the patient is maintained without affected by the suctioning force from the vacuum source. Since the catheter adaptor 104 is isolated, a rinsing region (not labeled) arranged between the knob 202 and the catheter adaptor 104 may be flushed via the flush tubing 104b, which increases the cleanliness and efficiency while effectively prevent backwash of secretions or cleaning solution to the patient end or to the environment during flushing. More importantly, while the rinsing region is being flushed via the flush tubing 104b, the side port 103 can continuously and hermetically supply oxygen to the patient through the interconnected patient tubing adaptor 102. In summary, the instant embodiment, despite the rotation of the knob 202, the side port 103 still continuously supplies oxygen to the patient through the patient tubing adaptor 102.

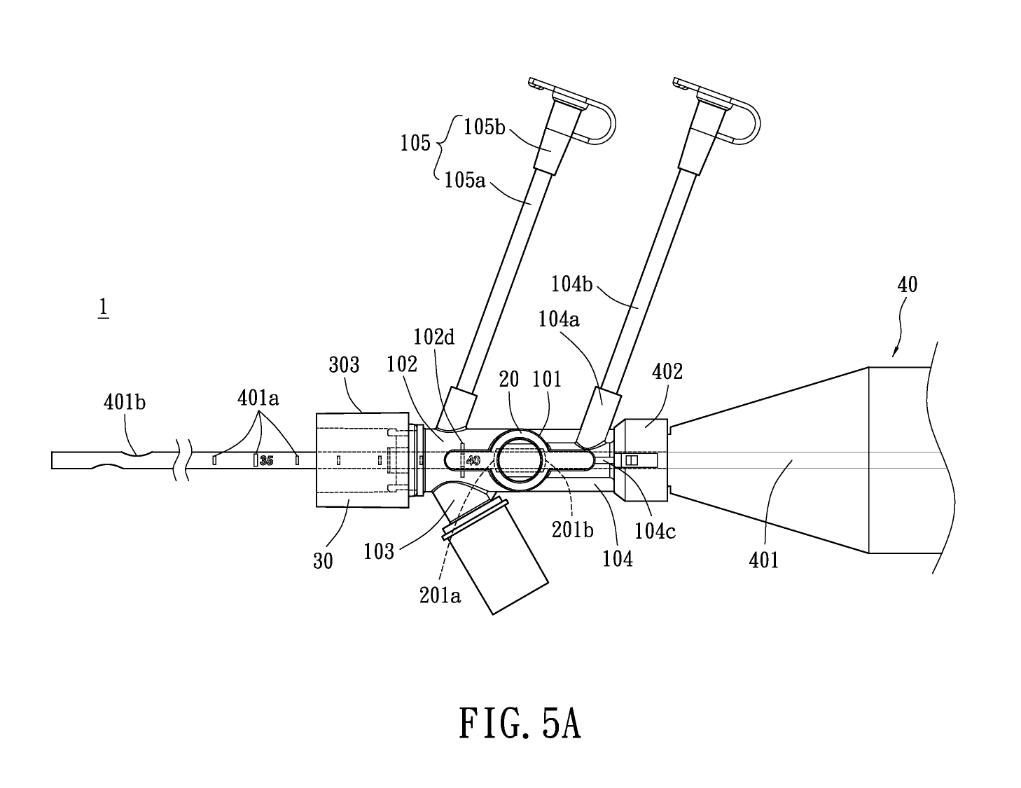

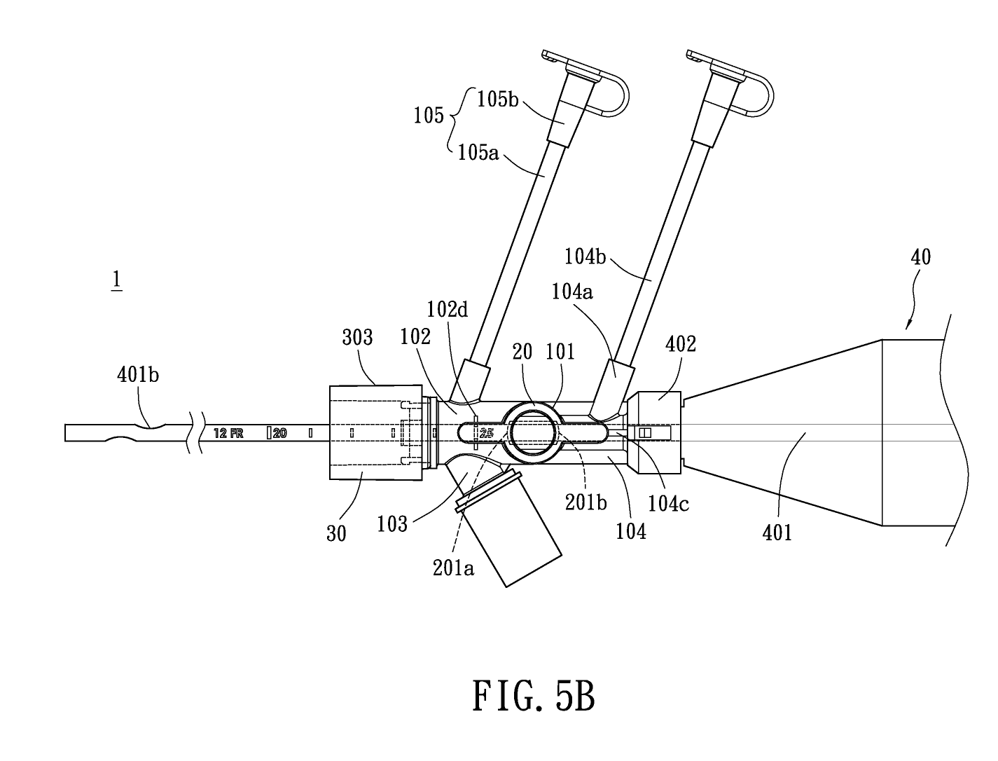

Referring to FIG. 5A and 5B, the switchable device 1 may also be applied with a catheter sleeve 40 engaged to the proximal end of the catheter adaptor 104. The catheter sleeve 40 includes a generally long flexible catheter 401 defined with at least one concaved opening 401b on the distal end of the catheter 401. In addition, the catheter 401 is formed with a plurality of depth markings 401a on the external surface thereof. Each of the depth markings 401a can be formed at a pre-determined distance therebetween as a printed mark. However, the formation of the depth markings 401a is not limited to the examples of the instant embodiment provided therein. The catheter 401 can be inserted through the device body 10 from the proximal end through the inline configuration towards the distal end. As the catheter 401 is advanced through the device body 10 towards the distal end, the indicator 102d is selectively paired with one of the depth markings 401a. Hence, medical staffs can selectively pair the desired depth marking 401a with the indicator 102d by viewing through the transparent patient tubing adaptor 102 and the transparent housing 303 of the patient end adaptor 30, thereby controlling the exact distance the catheter 401 is extended from the patient tubing adaptor 102 (not shown in figures).

Referring to FIG. 5A, as the catheter 401 is advanced from the catheter adaptor 104 towards the patient tubing adaptor 102, the indicator 102d is correspondingly paired with the depth marking 401a at an example value of 40. The example value of 40 represents the distal end of the catheter 401 defined with at least one concaved opening 401b is 40 inches away from the patient tubing adaptor 102. Similarly in FIG. 5B, as the indicator 102d is correspondingly paired with the depth marking 401a at an example value of 25. The example value of 25 represents the distal end of the catheter 401 defined with at least one concaved opening 401b is 25 inches away from the patient tubing adaptor 102. Therefore, through the application of the indicator 102d, the distance extending from the patient tubing adaptor 102 can be quickly and conveniently identified and the setup time can be significantly reduced. Furthermore, an injection port 105 may be formed on the patient tubing adaptor 102 for medication injections which removes secretion residue. The injection port 105 includes an injection tubing 105a, and an injection cap 105b disposing on one end of the injection tubing 105a.

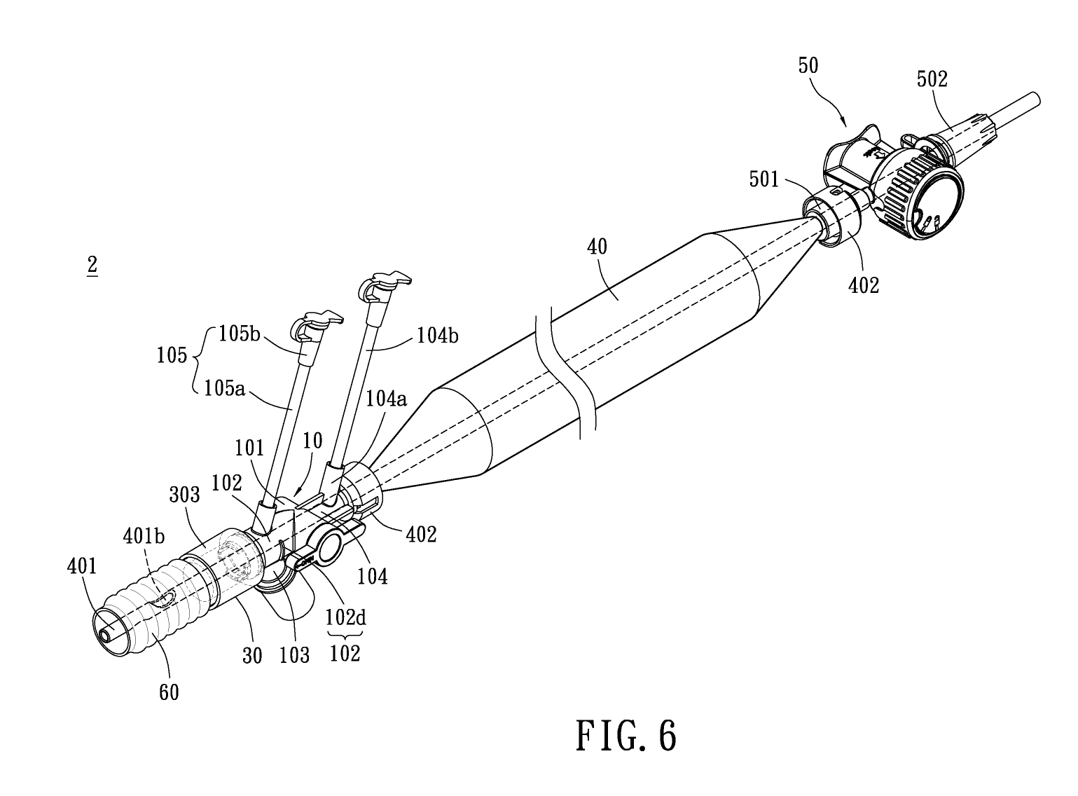

Turning to FIG. 6, the instant embodiment of instant disclosure also includes a closed suction catheter system 2 having a press control valve 50 and a corrugated tubing 60 coupled with the patient tubing adaptor 102. The catheter sleeve 40 is disposed between and engaged with the switchable device 1 and the press control valve 50 on opposite ends. Specifically, the catheter sleeve 40 also includes a seal bushing (not numbered), a scraper ring (not shown), and two collars 402, wherein each of the collars 402 secures the sleeve 40 with an inferential fit around the periphery of the press control valve 50 and the device 1. Thus, the catheter 401 can be advanced and drawn back from the airway of the patient through the corrugated tubing 60. In the instant embodiment, the seal bushing is a rubber material which seals off the catheter 401 to the environment, thus preventing medical staffs from exposure to the bacteria or viruses in secretions. The scraper ring is disposed between the collar 402 and the device 1 for scraping and removing secretions. The scraper ring is preferably fabricated from a rubber material.

Furthermore, the press control valve 50 includes a valve adaptor 501 and a suction adaptor 502 to facilitate medical staffs to perform the suction procedure. In addition, the patient tubing adaptor 102 may be coupled with an oxygenating machine (not shown in figures) to provide the patient with oxygen, and the injection port 105 may be formed with a scraper structure (not shown in figures) to scrap off and collect secretions.

In summary, the instant disclosure includes the device body 10 generally opened in two opposing ends and the two-way switch valve 20 correspondingly rotatable with respect to the body 10. With the misalignment design of the two-way switch valve 20 and the side port 103, the switchable device 1 provides a simple, easy to operate, and low cost structure for effectively control the opening and closing of fluid flow within, Moreover, the 360-degree freedom of rotation is provided by the structural elements such as the shoulder 102c and the groove 102a of the patient tubing adaptor 102. As a result, the freedom of rotation is suitable in various medical environments and prevents accidental suction loss caused by obstruction of flow when patients turn and flip. In addition, the instant disclosure also provides the indicator 102d which selectively pairs with one of the depth markings 401a. Hence, through the transparent patient tubing adaptor 102 and the transparent housing 303, the indicator 102d can be clearly and selectively paired with the desired depth marking 401a, thereby controlling the exact distance of the catheter 401 being advanced into an airway of the patient in a safe, comfortable, and bacterial infection reducing fashion. Besides clear pairing with the depth markings 401a, the indicator 102d also provides quick and convenient control of the exact distance of the catheter 401 extending from the patient tubing adaptor 102, thereby reducing setup time. Furthermore, whereby with the ribs 104c providing structural support to the radially retracted design of the catheter adaptor 104, cost is significantly reduced.

The figures and descriptions supra set forth illustrated the preferred embodiments of the instant disclosure; however, the characteristics of the instant disclosure are by no means restricted thereto. All changes, alternations, combinations or modifications conveniently considered by those skilled in the art are deemed to be encompassed within the scope of the instant disclosure delineated by the following claims.

What is claimed is:

1. A switchable device for closed suction catheters, comprising:
a device body integrally formed with
a valve sleeve defining a cavity that interconnects an opening arranged proximate to a top portion thereof;
a patient tubing adaptor formed on a sidewall of the valve sleeve and includes a groove formed on an end thereof for rotatably engaging a patient end adaptor;
a side port slantingly extending from and interconnecting the patient tubing adaptor; and
a catheter adaptor formed on the sidewall of the valve sleeve interconnected with the cavity; and
a two-way switch valve rotatably adapted to the device body including a stem and a knob, the stem disposed in the cavity defined by the valve sleeve, including a first opening and a second opening forming a channel to communicate flow therebetween, and the knob integrally formed with a plurality of handles and generally arranged on the apex of the stem.

2. The switchable device as recited in claim 1, wherein the end of the patient tubing adaptor having the groove is defined by the integral formation of a ring member and a plurality of shoulders extending from the ring member.

3. The switchable device as recited in claim 2, wherein the patient end adaptor includes a concentric outer layer and a concentric inner layer having a lip conformingly coupled with the groove for enabling the outer layer and inner layer to rotate freely with respect to the patient tubing adaptor.

4. The switchable device as recited in claim 3, wherein the patient tubing adaptor and the catheter adaptor provide an inline configuration to correspondingly interconnect the first and second opening with the patient tubing adaptor and the catheter adaptor.

5. The switchable device as recited in claim 4, wherein the catheter adaptor is integrally formed and interconnected with a flush port coupled with a flush tubing.

6. The switchable device as recited in claim 5, wherein two handles of the knob are correspondingly formed with respect to the patient tubing adaptor and the catheter adaptor.

7. The switchable device as recited in claim 6, wherein the patient end adaptor includes a transparent housing and the patient tubing adaptor includes an indicator having colored signage.

8. The switchable device as recited in claim 7, wherein the catheter adaptor includes a collar coupling an end of a catheter sleeve around the periphery of the catheter adaptor with an interferential fit, and a catheter formed with a plurality of depth marks and at least one concaved opening at an end of the catheter.

9. The switchable device as recited in claim 8, wherein the catheter is movable with respect to the collar, the catheter adaptor, and the patient tubing adaptor for the indicator to be selectively corresponded to the depth markings and the end of the catheter with at least one concaved opening to advance through the patient tubing adaptor.

10. The switchable device as recited in claim 9, wherein the catheter adaptor further includes at least two pairs of oppositely disposed ribs thereon and the patient tubing adaptor includes at least one injection port having an injection tubing and an injection cap for covering the injection tubing.

11. The switchable device as recited in claim 1, wherein the catheter adaptor further includes at least two pairs of oppositely disposed ribs thereon and the patient tubing adaptor includes at least one injection port having an injection tubing and an injection cap for covering the injection tubing.

12. A closed suction catheter system, comprising:
a switchable device for closed suction catheters, comprising:
a device body integrally formed with
a valve sleeve defining a cavity that interconnects an opening arranged proximate to a top portion thereof,
a patient tubing adaptor formed on a sidewall of the valve sleeve and includes a groove formed on an end thereof for rotatably engaging a patient end adaptor, wherein the patient tubing adaptor includes an indicator having color signage,
a side port slantingly extended from and interconnected with the patient tubing adaptor,
a catheter adaptor formed on the sidewall of the valve sleeve interconnected with the cavity; and
a patient end adaptor having a transparent housing rotatably coupled to the grooved end of the patient tubing adaptor,
a two-way switch valve rotatably adapted to the device body including a stem and a knob, the stem disposed in the cavity defined by the valve sleeve, including a first opening and a second opening forming a channel to communicate flow therebetween, and the knob integrally formed with a plurality of handles and generally arranged on the apex of the stem;
a catheter sleeve coupled with the catheter adaptor at an end thereof including a catheter having a plurality of depth markings disposed therein for selectively corresponding with the indicator and advancing an end of a catheter through the patient tubing adaptor; and
a press valve control disposed on a second end of the catheter sleeve opposite from the catheter adaptor.

13. The closed suction catheter system as recited in claim 12, wherein the end of the patient tubing adaptor having the groove is defined by the integral formation of a ring member and a plurality of shoulders extending from the ring member.

14. The closed suction catheter system as recited in claim 13, wherein the patient end adaptor includes a concentric outer layer and a concentric inner layer having a lip conformingly coupled with the groove for enabling the outer layer and inner layer to rotate freely with respect to the patient tubing adaptor.

15. The closed suction catheter system as recited in claim 14, wherein the patient tubing adaptor and the catheter adaptor provide an inline configuration to correspondingly interconnect the first and second opening with the patient tubing adaptor and the catheter adaptor, and the knob includes two handles correspondingly formed with respect to the patient tubing adaptor and the catheter adaptor.

16. The closed suction catheter system as recited in claim 15, wherein the catheter sleeve includes a collar disposed on the catheter adaptor, the catheter is formed with at least one concaved opening at an end thereof, and the catheter is movable with respect to the collar, the catheter adaptor, and the patient tubing adaptor.

17. The closed suction catheter system as recited in claim 16, wherein the catheter adaptor further includes at least two oppositely disposed ribs thereon and the patient tubing adaptor further includes at least one injection port having an injection tubing and an injection cap for covering the injection tubing.

18. The closed suction catheter system as recited in claim 12, wherein the catheter adaptor further includes at least two oppositely disposed ribs thereon and the patient tubing adaptor further includes at least one injection port having an injection tubing and an injection cap for covering the injection tubing.
